(19) United States
(12) Patent Application Publication
(10) Pub. No.: US 2010/0330169 A1
(43) Pub. Date: Dec. 30, 2010

(54) Pharmaceutical Tablet Containing A Liquid Filled Capsule

(76) Inventors: Frank Bunick, Randolph, NJ (US); Leo B. Kriksunov, Glenside, PA (US); Joseph Luber, Quakertown, PA (US); Harry Sowden, Glenside, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/824,311

(22) Filed: Jun. 28, 2010

Related U.S. Application Data
(60) Provisional application No. 61/221,182, filed on Jun. 29, 2009.

(51) Int. Cl. A61K 9/48; A61P 11/10; A61P 11/02; A61P 37/08; A61P 23/00

(52) U.S. Cl. 424/452

(57) ABSTRACT

In one aspect, the present invention features a tablet including a compressed core and a liquid filled capsule, wherein the compressed core includes a first pharmaceutically active agent, the compressed core has a cavity exposed on the surface of the core, and the capsule is contained within the cavity such that a portion of the capsule is visible on the surface of the tablet, wherein the capsule has a diameter of at least 500 microns.

### CROSS REFERENCE TO RELATED APPLICATIONS

This application claims priority of the benefits of U.S. Provisional Application Ser. No. 61/221,182 filed Jun. 29, 2009. The complete disclosure of the aforementioned related U.S. patent application is hereby incorporated by reference for all purposes.

### BACKGROUND OF THE INVENTION

Compressed tablets are known as one of the most cost effective, consumer friendly and convenient dosage forms available for delivering pharmaceutically active agents. However, necessary ingredients (e.g., pharmaceutically active agents) can be incompatible when combined in a tablet (e.g., ingredients which react together and/or need to be administered in liquid form). Further, sequential delivery (e.g., local and systemic delivery) of pharmaceutically active agents or sensory ingredients (e.g., sensates and flavoring) in a single tablet can also difficult to achieve. It can also be advantageous to visually communicate to a consumer or patient that a delivery form may deliver two types of active pharmaceutically active agents. In addition, some pharmaceutically active agents are preferentially delivered in liquid form either due to improved solubility or improved stability in aqueous or lipid base materials, and in some instances, result in improved absorption rates in the gastrointestinal system.

The present invention relates to a tablet the contains both a compressed core and a liquid filled capsule, which can be used to hold ingredients that are best administered in liquid form and/or can be administered locally to the mouth or throat.

### SUMMARY OF THE INVENTION

In one aspect, the present invention features a tablet including a compressed core and a liquid filled capsule, wherein the compressed core includes a first pharmaceutically active agent, the compressed core has a cavity exposed on the surface of the core, and the capsule is contained within the cavity such that a portion of the capsule is visible on the surface of the tablet, wherein the capsule has a diameter of at least 500 microns.

In one aspect, the present invention features a method of manufacturing such a tablet by the steps of: (a) adding a powder including a pharmaceutically-acceptable carrier and the first pharmaceutically active agent to a tablet die; (b) compressing the powder within the tablet die to form the compressed core; and (c) inserting the capsule into the cavity in the compressed core to form the tablet.

Other features and advantages of the present invention will be apparent from the detailed description of the invention and from the claims.

### BRIEF DESCRIPTION OF THE FIGURES

FIG. 1A is a perspective view of tablet 10 having a compressed core 20 and a liquid filled capsule 50.

FIG. 1B is a cross-section view along line 1B-1B′ of tablet 10 having a compressed core 20 and a liquid filled capsule 50.

FIG. 2A is a perspective view of tablet 10 having a compressed core 20 and liquid filled capsules 50 and 55.

FIG. 2B is a cross-section view along line 2B-2B′ of tablet 10 having a compressed core 20 and liquid filled capsules 50 and 55.

FIG. 3 is a cross section view of a tablet having a compressed core 20, a liquid filled capsule 50, and a transparent film 90.

### DETAILED DESCRIPTION OF THE INVENTION

It is believed that one skilled in the art can, based upon the description herein, utilize the present invention to its fullest extent. The following specific embodiments can be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

As discussed above, the present invention features a tablet including a compressed core and a liquid filled capsule, wherein the compressed core includes a first pharmaceutically active agent, the compressed core has a cavity exposed on the surface of the core, and the capsule is contained within the cavity such that a portion of the capsule is visible on the surface of the tablet. The benefits of the above tablet include, but are not limited to, (i) the separation of incompatible pharmaceutically active agents (or other ingredients) with the visual communication of such separation to the consumer, (ii) the ability to place certain active ingredients which are more liquid stable (e.g., aqueous or lipid) into solid tablet form, (iii) the ability to place certain pharmaceutically active agents that display improved dissolution or absorption in liquid form into a solid tablet form (e.g., the pharmaceutically active agent may not require further dissolution in a gastric liquid medium or may allow for faster emptying of the active from the stomach to the duodenum and small intestine where the agent is absorbed), and (iv) the ability to have differentiated dissolution profiles within a single tablet whereby the liquid filled capsule can have a different dissolution profile as compared to the compressed core (e.g., the capsule can be coated to delay dissolution of the capsule and consequently absorption of the pharmaceutically active agents contained within the capsule or conversely the capsule can be adapted to dissolve faster as compared to the compressed core which, in one example, can comprise coated particles delaying the release of the pharmaceutically active agents from the compressed core).

In one embodiment, the capsule contains a pharmaceutically active agent, which may be the same or a different pharmaceutically active agent than the pharmaceutically active agent contained within the compressed core.

### Manufacture of Compressed Core

As discussed above, in one embodiment, the present invention features a method of manufacturing a tablet of the present invention including the steps of adding a powder including a pharmaceutically-acceptable carrier and the first pharmaceutically active agent to a tablet die (i.e., a die cavity) and compressing the powder within the tablet die to form the compressed core. In one embodiment of the invention, the powders having an average particle size of about 50 microns to about 500 microns, such as between 50 microns and 300 microns. Particles in this size range are particularly useful for direct compression processes.

In embodiment, the components of powder are blended together, for example as dry powders, and fed into the tablet die of an apparatus that applies pressure to form a tablet. Any suitable compacting apparatus may be used, including, but not limited to, conventional unitary or rotary tablet press. In one embodiment, the tablet may be formed by compaction using a rotary tablet press (e.g., such as those commercially available from Fette America Inc., Rockaway, N.J., or Manesty Machines LTD, Liverpool, UK). In general, a metered volume of powder is filled into a tablet die, where the powder is either gravity fed or mechanically fed from a feeder, of the rotary tablet press, and the cavity rotates as part of a “die table” from the filling position to a compaction position. At the compaction position, the powder is compacted between an upper and a lower punch, then the resulting tablet is pushed from the tablet die by the lower punch and then guided to an injection chute by a stationary “take-off” bar. Advantageously, the direct compression process enables the minimization or elimination of water-soluble, non-saccharide polymeric binders such as polyvinyl pyrrolidone, alginates, hydroxypropyl cellulose, hydroxypropylmethylcellulose, hydroxyethylcellulose, which could have a negative effect on dissolution.

In another embodiment, the tablet may be prepared by the compression methods and apparatus described in United States Patent Application Publication No. 20040156902. Specifically, the tablet may be made using a rotary compression module including a fill zone, insertion zone, compression zone, ejection zone, and purge zone in a single apparatus having a double row die construction. The dies of the compression module may then be filled using the assistance of a vacuum, with filters located in or near each die. The purge zone of the compression module includes an optional powder recovery system to recover excess powder from the filters and return the powder to the dies.

In another embodiment, the tablet may be prepared by a wet-granulation method, in which the excipients and a solution or dispersion of a wet binder (e.g., an aqueous cooked starch paste or solution of polyvinyl pyrrolidone) are mixed and granulated. Suitable apparatus for wet granulation include low shear mixers (e.g., planetary mixers), high shear mixers, and fluid beds (including rotary fluid beds). The resulting granulated material may then be dried, and optionally dry-blended with further ingredients (e.g., excipients such as, for example, lubricants, colorants, and the like). The final dry blend is then suitable for compression by the methods described in the previous paragraph. Methods for direct compression and wet granulation processes are known in the art.

In one embodiment, the tablet is prepared by the compression methods and apparatus described in issued U.S. Pat. No. 6,767,200, the disclosure of which is incorporated herein by reference. Specifically, the tablet is made using a rotary compression module including a fill zone, compression zone, and ejection zone in a single apparatus having a double row die construction as shown in FIG. 6 therein. The dies of the compression module are preferably filled using the assistance of a vacuum, with filters located in or near each die.

In one embodiment of the invention, the tablet may be a directly compressed tablet made from a powder that is substantially free of water-soluble polymeric binders and hydrated polymers. As used herein, what is meant by “substantially free” is less than 5%, such as less than 1%, such as less than 0.1%, such as completely free (e.g., 0%). This composition is advantageous for maintaining an immediate release dissolution profile, minimizing processing and material costs, and providing for optimal physical and chemical stability of the tablet. In one embodiment, the density of the tablet is greater than about 0.9 g/cc.

The tablet may have one of a variety of different shapes. For example, the tablet may be shaped as a polyhedron, such as a cube, pyramid, prism, or the like; or may have the geometry of a space figure with some non-flat faces, such as a cone, truncated cone, cylinder, sphere, torus, or the like. In certain embodiments, a tablet has one or more major faces. For example, the tablet surface typically has opposing upper and lower faces formed by contact with the upper and lower punch faces in the compression machine. In such embodiments the tablet surface typically further includes a “belly-band” located between the upper and lower faces, and formed by contact with the die walls in the compression machine. A tablet may also be a multilayer tablet.

### Powder

As discussed above, the tablet is manufactured by compressing a powder containing a pharmaceutically-acceptable carrier. The carrier may contain one or more suitable excipients for the formulation of tablets. Examples of suitable excipients include, but are not limited to, fillers, adsorbents, binders, disintegrants, lubricants, glidants, release-modifying excipients, sweeteners, superdisintegrants, flavor and aroma agents, antioxidants, texture enhancers, and mixtures thereof.

Suitable fillers include, but are not limited to, water-soluble compressible carbohydrates such as sugars (e.g., dextrose, sucrose, maltose, and lactose), starches (e.g., corn starch), sugar-alcohols (e.g., mannitol, sorbitol, maltitol, and xylitol), starch hydrolysates (e.g., dextrins, and maltodextrins), and water insoluble plastically deforming materials (e.g., microcrystalline cellulose or other cellulosic derivatives), and mixtures thereof.

Suitable adsorbents include, but are not limited to, water-insoluble adsorbents such as dicalcium phosphate, tricalcium phosphate, silicified microcrystalline cellulose (e.g., such as distributed under the PROSOLV brand (PenWest Pharmaceuticals, Patterson, N.Y.)), magnesium aluminometasilicate (e.g., such as distributed under the NEUSILIN brand (Fuji Chemical Industries (USA) Inc., Robbinsville, N.J.), clays, silicas, bentonite, zeolites, magnesium silicates, hydrotalcite, veegum, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof.

Suitable glidants include, but are not limited to, colloidal silicon dioxide.

Suitable release-modifying excipients include, but are not limited to, swellable erodible hydrophilic materials, insoluble edible materials, pH-dependent polymers, and mixtures thereof.

Suitable swellable erodible hydrophilic materials for use as release-modifying excipients include, but are not limited to, water swellable cellulose derivatives, polyalkylene glycols, thermoplastic polyalkylene oxides, acrylic polymers, hydrocolloids, clays, gelling starches, swelling cross-linked polymers, and mixtures thereof. Examples of suitable water swellable cellulose derivatives include, but are not limited to, sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose, and mixtures thereof. Examples of suitable polyalkylene glycols include, but are not limited to, polyethylene glycol. Examples of suitable thermoplastic polyalkylene oxides include, but are not limited to, poly (ethylene oxide). Examples of suitable acrylic polymers include, but are not limited to, potassium methacrylatedivinylbenzene copolymer, polymethylmethacrylate, high-molecular weight cross-linked acrylic acid homopolymers and copolymers commercially available from Noveon Chemicals under the tradename, “CARBOPOL” (e.g., having a viscosity of greater than 50,000 centipoise when tested using a Brookfield RVT Viscometer at 25° C., using spindle #7, when dispersed in a basic solution). Examples of suitable hydrocolloids include, but are not limited to, alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan, and mixtures thereof. Examples of suitable clays include, but are not limited to, smectites such as bentonite, kaolin, and laponite; magnesium trisilicate; magnesium aluminum silicate; and mixtures thereof. Examples of suitable gelling starches include, but are not limited to, acid hydrolyzed starches, swelling starches such as sodium starch glycolate and derivatives thereof, and mixtures thereof. Examples of suitable swelling cross-linked polymers include, but are not limited to, cross-linked polyvinyl pyrrolidone, cross-linked agar, and cross-linked carboxymethylcellulose sodium, and mixtures thereof.

Suitable insoluble edible materials for use as release-modifying excipients include, but are not limited to, water-insoluble polymers and low-melting hydrophobic materials, copolymers thereof, and mixtures thereof. Examples of suitable water-insoluble polymers include, but are not limited to, ethylcellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their salts, and mixtures thereof. Examples of suitable fatty acid esters include, but are not limited to, sucrose fatty acid esters, mono-, di-, and tri-glycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof.

Suitable pH-dependent polymers for use as release-modifying excipients include, but are not limited to, enteric cellulose derivatives such as hydroxypropyl methylcellulose phthalate, hydroxypropyl methylcellulose acetate succinate, cellulose acetate phthalate; natural resins such as shellac and zein; enteric acetate derivatives such as polyvinylacetate phthalate, cellulose acetate phthalate, acetaldehyde dimethylcellulose acetate; and enteric acrylate derivatives such as polymethacrylate-based polymers such as poly(methacrylic acid, methyl methacrylate) 1:2 (which is commercially available from Rohm Pharma GmbH under the tradename EUDRAGIT S), and poly(methacrylic acid, methyl methacrylate) 1:1 (which is commercially available from Rohm Pharma GmbH under the tradename EUDRAGIT L), and mixtures thereof.

Examples of suitable sweeteners include, but are not limited to, synthetic or natural sugars, sucralose, saccharin, sodium saccharin, aspartame, acesulfame K or acesulfame, potassium acesulfame, thaumatin, glycyrrhizin, dihydrochalcone, alitame, miraculin, monellin, stevside, and mixtures thereof.

Examples of superdisintegrants include, but are not limited to, croscarmellose sodium, sodium starch glycolate and cross-linked povidone (crospovidone). In one embodiment the tablet contains up to about 5% by weight of such superdisintegrant.

Examples of suitable flavor and aroma agents include, but are not limited to, essential oils including distillations, solvent extractions, or cold expressions of chopped flowers, leaves, peel or pulped whole fruit containing mixtures of alcohols, esters, aldehydes and lactones; essences including either diluted solutions of essential oils, or mixtures of synthetic chemicals blended to match the natural flavor of the fruit (e.g., strawberry, raspberry, and black currant); artificial and natural flavors of brews and liquors (e.g., cognac, whisky, rum, gin, sherry, port, and wine); tobacco, coffee, tea, cocoa, and mint; fruit juices including expelled juice from washed, scrubbed fruits such as lemon, orange, and lime; mint; ginger; cinnamon; cacoe/cocoa; vanilla; liquorice; menthol; eucalyptus; aniseeds nuts (e.g., peanuts, coconuts, hazelnuts, chestnuts, walnuts, and colanuts); almonds; raisins; and powder, flour, or vegetable material parts including tobacco plant parts (e.g., the genus Nicotiana in amounts not contributing significantly to a level of therapeutic nicotine), and mixtures thereof.

Examples of antioxidants include, but are not limited to, tocopherols, ascorbic acid, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzoic acid, and sorbic acid, and mixtures thereof.

Examples of texture enhancers include, but are not limited to, pectin, polyethylene oxide, and carrageenan, and mixtures thereof. In one embodiment, texture enhancers are used at levels of from about 0.1% to about 10% percent by weight.

### Formation of Cavity

As discussed above, the compressed core has one or more cavities to hold the one or more liquid filled capsules. In one embodiment, the one or more cavities are formed by the tablet die during compression of the compressed core (e.g., the tooling is shaped in such a way, such as core rod tooling, in order to form a cavity).

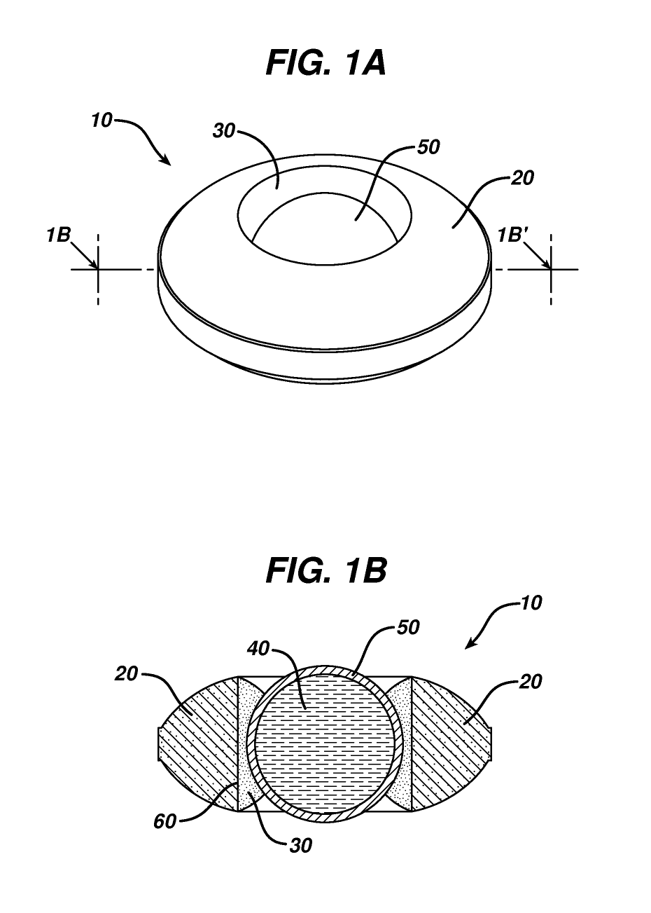

In one embodiment, the cavity or cavities extend through the compressed tablet from one face to the opposite face of tablet. For example, as shown in FIG. 1A and FIG. 1B, tablet 10 has a compressed core 20 with a single cavity having a circular cavity wall 60. Liquid filled capsule 50 (filled with liquid 40) is held within the cavity with adhesive 30.

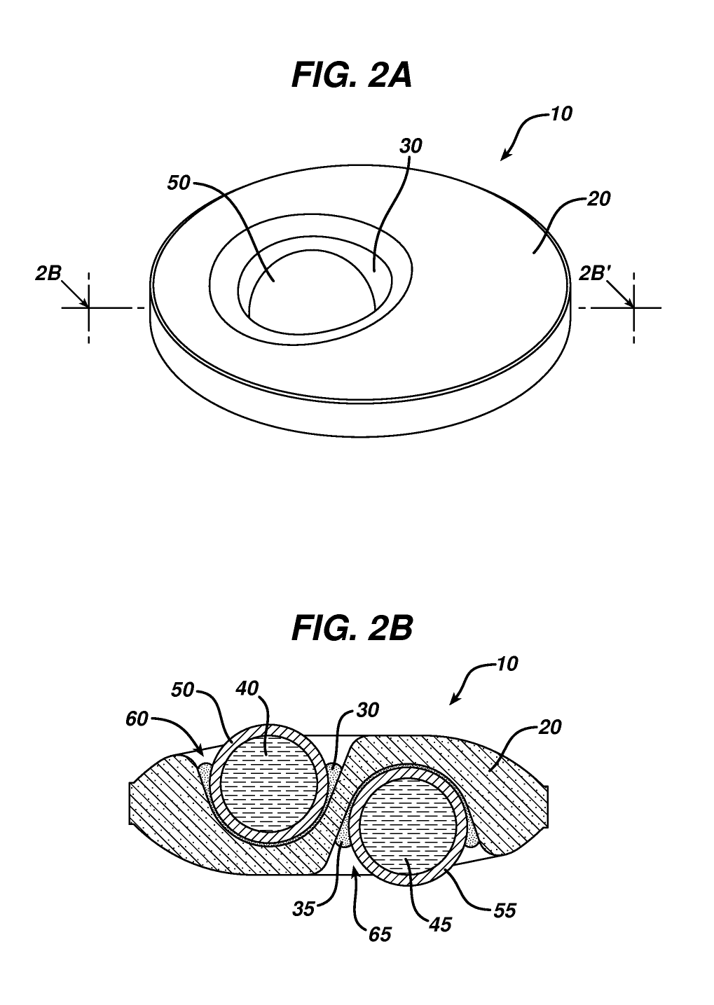

In another embodiment, the cavity or cavities only partially extends through the tablet. For example, as shown in FIGS. 2A and 2B, the tablet 10 having compressed core 20 has two cavities, defined by cavity walls 60 and 65, respectively. These cavities are both off-set from the center of longest axis of the tablet. Liquid filled capsules 50 (filled with liquid 40) and liquid filled capsule 55 (filled with liquid 45) are held, respectively, with adhesive 30 and adhesive 35.

In one embodiment, the compressed tablet is a multilayer tablet, e.g. a bilayer tablet or a trilayer tablet. In one embodiment wherein the tablet is a bilayer tablet, the first layer includes a cavity for a first liquid filled capsule. In one embodiment wherein the tablet is a bilayer tablet, the first layer includes a cavity for a first liquid filled capsule and the second layer includes a cavity for a second liquid filled capsule.

### Assembly of Tablet

In one embodiment, a pick-and-place system is used wherein the capsule is mechanically placed into the cavity. In one embodiment the capsule fits snugly in the cavity so as to prevent substantially any movement of the capsule within the cavity, with the diameter of the cavity being substantially equal to the diameter of the capsule, thus immobilizing the capsule within the cavity. In one embodiment, the capsule is affixed to the cavities with an edible adhesive material in order to affix the liquid filled capsule to the compressed portion. In one embodiment, the edible adhesive-like material includes an ingredient selected from the group consisting of gelatin, polyethylene glycol, polyethylene oxide, polycaprolactone, carnuba wax, microcrystalline wax, oppanol, shellac wax, and beeswax. In one embodiment, the cavity surface is coated by a film coat using spray coating methods known in the art. In another embodiment, the cavity surface is dip coated, for example with gelatin coating.

In one embodiment, the edible adhesive material is pre-melted (e.g., at a temperature from about 35° C. to about 100° C.) and then added to cavity. In another embodiment, the adhesive is added to the cavity as a powder and heated later.

In one embodiment, the compressed core contains excipients having low temperature of melting, such as polyethylene glycol (PEG) or a wax. After the capsule is placed into the cavity, the resulting assembly is exposed to heat sufficient to melt a portion of the low melting excipient inside the cavity, which then upon cooling develops a bond with the capsule at the capsule/compressed core interface within the cavity.

In another embodiment, the adhesive is applied by first mixing a sugar and/or a polymer (e.g., polymethacrylates, hydroxypropylmethylcellulose, hydroxypropylcellulose, starch, and polyvinyl pyrrolidone) in water solution and then placing about 0.1 mL to about 5 mL of the solution (e.g., prepared at about 1 percent to about 50 percent solids) to the cavity. The liquid filled capsule is then placed in the cavity and then the tablet is allowed to dry. In one embodiment, the adhesive is water, which is added to the cavity and bonds the capsule to the compressed core by dissolving a small portion of the core and/or capsule wall, with the bond developing after the tablet is allowed to dry.

In one embodiment, the adhesive is applied by mixing a polymer to an organic solvent solution, and the resulting solution is applied in the manner described above for an aqueous solution. In one embodiment, the polymer hydroxypropylcellulose is prepared in a solution of ethanol, methanol, isopropanol, or mixtures thereof.

In one embodiment, the edible adhesive material can be added to the tablet in an amount from about 0.05 percent to about 40 percent, e.g. from about 0.5 percent to about 10 percent, by weight of the total weight of the tablet.

In one embodiment, the edible adhesive material which is used to fixate the capsule within the cavity contains an effervescent material, such as carbonate salts (e.g., calcium carbonate or sodium bicarbonate). In such an embodiment, upon exposure of the effervescent materials to acidic environment (e.g. to media having pH from about 1 to 3, more preferably to pH about 2), the carbonate salt in the adhesive reacts with the acidic media, forming carbon dioxide bubbles, and releasing the capsule from the cavity into the media. Optionally, the edible adhesive material may also contain acidic component which will accelerate the evolution of carbon dioxide bubbles or actuate the effervescent materials upon exposure to aqueous media with neutral pH. In certain embodiments, the release or launch of the capsule from the cavity and also the effervescent effects of the carbon dioxide bubbles emitted from the cavity can impart a rotational momentum on the dose form, particularly when the cavity is located offset from the center of the compressed core. The rotational momentum can provide for accelerated dissolution of the dose form.

In one embodiment, the liquid filled capsule is incorporated into the core when the core is being compressed. In this embodiment, the capsule is deposited into the mold with the granulation and then subject to compression at low to medium compression forces to avoid bursting of the capsule. Capsule is thus embedded into the core. Adding the capsule to the mold before the mold is filled with the powder, or after the mold is filled with the powder, ensures that at least a portion of the capsule is visible on the surface of the resulting dose form.

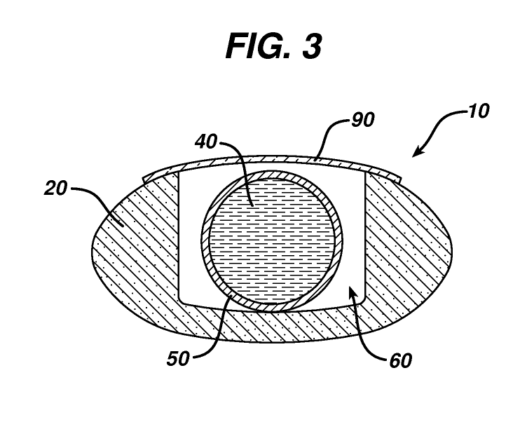

In one embodiment, the liquid filled capsule 50 is deposited into the cavity and is held in the cavity by transparent film 90 that is covering the cavity 60, e.g., as shown in FIG. 3. In one embodiment, transparent film 90 is clear. Edible transparent films are known in the art, and they can be adhered to the surface of the tablet using heat, adhesive, or moisture. In one embodiment the edible film is self-adhesive. In one embodiment, the capsule is fully contained in the cavity with the film covering the mouth of the cavity (not shown). In another embodiment, the capsule is protruding from the cavity and is over coated by the transparent film as in FIG. 3.

In one embodiment, the resulting dose form is further reinforced by a coating, such as spray or dip coating, or by exposure to heat that further sinters the dose form which optionally contains excipients having low temperature of melting, such as polyethylene glycol (PEG).

### Liquid-Filled Capsule

As set forth above, the tablet of the present invention includes one or more liquid filled capsules. What is meant by a “liquid filled capsule” is a capsule that has a shell containing a core that is a liquid.

The shell of the liquid filled capsule may be made of a variety of materials, including but not limited to: film forming materials, such as gelatin, gellan gum, hypromellose, starch, modified starch, and pectin; gums and viscosity modifiers such as xanthan gum, locust bean gum, and guar gum; plasticizers such as polyethylene glycol; propylene glycol; glycerin; sorbitol; triethyl citrate; tributyl citrate; dibutyl sebecate; vegetable oils such as castor oil, rape oil, olive oil, and sesame oil; surfactants such as polysorbates, sodium lauryl sulfates, and dioctyl-sodium sulfosuccinates; acetates of glycerol such as mono-, di-, and triacetates of glycerol; triacetin; acetyltributyl citrate; diethyloxalate; diethylmalate; diethyl fumarate; diethylmalonate; dioctylphthalate; dibutylsuccinate; glyceroltributyrate; hydrogenated castor oil; fatty acids; substituted triglycerides and glycerides; and mixtures thereof.

The liquid contained within the shell of the capsule may contain a variety of materials including solubilizers, carriers, viscosity modifiers, and pH adjusting materials (such as alkalizing agents, acids or buffering agents). The solubilizers and carriers may be aqueous or lipid based. The pharmaceutically active agents that may be contained in the liquid core may be solubilized or suspended in the liquid. Suitable solubilizers and carriers include, but are not limited to, vegetable oils, vegetable oil triglycerides and triacylglycerols (such as corn oil); polyglycolized glycerides (such as lauryl macrogol 32-glycerides and steroyl macrogol 32-glycerides including those sold under the tradename Gelucire® 44/14 and Gelucire® 50/13 available from the Gattefosse USA Corporation in Paramus, N.J., glycerol esters of fatty acids (such as those sold under the tradename Gelucire® 33/01, Gelucire® 39/01, and Gelucire® 43/01 available from the Gattefosse Corporation); neutral oils and triglycerides (such as medium chain triglycerides, fractionated coconut oil, caprylic and capric triglycerides. polyethylene glycol and polyoxyethylene stearates (such as polyethylene glycol 15 hydroxystearate as sold under the tradename Solutol® HS 15 available from the BASF Corporation in Florham Park, N.J.; vegetable, soybean and egg yolk lecithin (such as phosphatidyl choline and 1,2-diacyl-sn-glycero-3-phosphoryl choline such as those sold under the tradename Phospholipon® 90 G available from the American Lecithin Company in Oxford, Conn.; lecithin combined in propylene glycol (such as standardized mixtures of phosphatidylcholine, propylene glycol, mono- and di-glycerides, ethanol, soya fatty acids and ascorbyl palmitate, such as those sold under the tradename of Phosal® 50 PG available from the American Lechitin Coporation in Oxford, Conn.; capryl-caproyl macrogol-8-glycerides (such as those sold under the tradename Labrasol® available from the Gattefosse Corporation in Paramus, N.J.; polyethoxylated hydrogenated castor oil (such as glycerol-polyethylene glycol oxystearate, such as those sold under the tradename Cremophor® RH 40 and Cremophor® EL available from the BASF Coporation).

Examples of the pH adjusting agent in the liquid fill include, but are not limited to: alkalizing agent selected from the group consisting of alkalizing agents such as potassium hydroxide, sodium hydroxide, magnesium hydroxide, calcium hydroxide, potassium acetate, sodium acetate, magnesium acetate, calcium carbonate, calcium oxide, calcium phosphates, magnesium carbonate, magnesium oxide, magnesium phosphates, magnesium hydroxide carbonate, magnesium aluminum silicate, magaldrate, bentonite, zeolites, magnesium silicates, hydrotalcite, dihydroxyaluminum sodium carbonate, ammonium hydroxide, ammonium bicarbonate, ammonium carbonate, ethanolamine, diethanolamine, triethanolamine, sodium bicarbonate, potassium bicarbonate, magnesium hydroxide, aluminum hydroxide, magnesium phosphates, tetrasodium ethylenediaminetetraacetic acid and its hydrates; and acids such as citric acid, maleic acid, fumaric acid, phosphoric acid, and ascorbic acid.

In some embodiments a preservative agent may be added to the liquid fill, which include, but not limited to, sodium benzoate, methyl paraben, butyl paraben and propyl paraben.

In some embodiments, a viscosity modifier is added to the liquid fill, which include, but are not limited to, xanthan gum, carrageenan, hypromellose, guar gum, locust bean gum, and hydroxypropyl cellulose.

The capsule formulations can also include other suitable additives such as preservatives and/or coloring agents which are utilized to stabilize the capsule and/or impart a specific characteristic such as color or look to the capsule. The capsule may also contain flavorants, sensates, salivation inducing agents, fragrances, acidulants such as citric, fumaric or malic acid; cooling agents such as menthol or non-volatile coolers; and sweeteners such as but not limited to sucralose, aspartame, saccharine, acesulfame potassium and related salts and derivatives thereof.

In one embodiment, the capsule has a diameter (i.e., the largest diameter) of at least 500 microns, such as from about 500 microns to about 10,000 microns, e.g. from about 500 microns to about 5000 microns.

In one embodiment, the capsule includes ingredients selected from the group consisting of a second pharmaceutically active agent, a lubricant, a salivation-inducing agent, a warming sensate, a cooling sensate, and flavors.

Examples of lubricants include, but are not limited to, fatty acids, lechitin, silica oil, olive oil, mineral oil, and cocoa butter.

Examples of salivation-inducing agents include, but are not limited to, pilocarpine; N,N-disubstituted-2-phenylcyclopropylamines; spirooxathiolane-quinnuclidine; Heliopsis longpipes root; cholinesterase inhibitors, alkenecarboxylic acid N-alkylamides, trans-pellitorin, Succulence® sensates commercially available from International Flavors & Fragrances in Hazlet, N.J. and mixtures thereof. Examples of tingling sensates include, but are not limited to, Jambu Oleoresin, Zanthoxylum peperitum saanshool-I, spilanthol, sanshool, hydroxy sanshool, and pellitorin.

Examples of warming sensates include, but are not limited to, capsaicin, piperine, dihydrocapsaicin, chavicine, nonivamide, cis-pellitorine, ethyl ether, vanillyl propyl ether, vanillin propylene glycol acetal, ethyl vanillin propylene glycol acetal, gingerol, vanillyl butyl ether, 4-(I-menthoxy-methyl)-2-phenyl-1,3-dioxolane, 4-(I-menthoxy-methyl)-2-(3′,4′-dihydroxy-phenyl)-1,3-dioxolane, 4-(I-menthoxy-methyl)-2-(2′-hydroxy-3′-methoxy-phenyl)-1,3-dioxolane, 4-(I-menthoxy-methyl)-2-(4′-methoxyphenyl)-1,3-dioxolane, 4-(I-menthoxy-methyl)-2-(3′4′-methylenedioxy-phenyl)-1,3-dioxolane, hot pepper oil, capsicum oleoresin, ginger oleoresin, nonyl acid vanillylamide, and 4-(I-menthoxy-methyl)-2-(3′-methoxy-4′-hydroxyphenyl)-1,3-dioxolane.

Examples of cooling sensates include, but are not limited to, isopulegole, 3-(I-menthoxy)propan-1 2-diol, p-menthan-3,8-diol, 6-isopropyl-9-methyl-1,4-dioxaspiro-(4,5)-decane-2-methanol, menthyl succinate, alkaline earth salts of menthyl succinate, trimethyl cyclohexanol, N-ethyl-2-isopropyl-5-methylcyclohexane carboxamide, 3-(I-menthoxy)-2-methyl-propan-1,2-diol, mint oil, peppermint oil, wintergreen, menthone, menthone glycerin ketal, menthyl lactate, [1′R,2′S,5′R]-2-(5′-methyl-2′-(methylethyl)cyclohexyloxy)ethan-1-ol, [1′R,2′S,5′R]-3-(5′-methyl-2′-(methylethyl)cyclohexyloxy)propan-1-ol, [1′R,2′S,5′R]-4-(5′-methyl-2′-(methylethyl)cyclohexyloxy)butan-1-ol, spearmint, gardamide, N-substituted p-menthane carboxamides, menthoxypropan-1,2-diol, menthol and menthyl esters, such as Cooler #2® which is available from International Flavors & Fragrances in Hazlet, N.J.

In one embodiment, the capsule includes a second pharmaceutically active agent. In one embodiment, the second pharmaceutically active agent is the same as the first pharmaceutically active agent. In one embodiment, the second pharmaceutically active agent is different from the first pharmaceutically active agent.

In one embodiment, the liquid filled capsule is round. In one embodiment, more than one capsule is used, e.g. 2 capsules and 3 capsules are contained in one tablet. In one embodiment, at least 10 percent, e.g. at least 20 percent or at least 40 percent, of the surface of the capsule is visible (e.g., exposed on the surface of the tablet). In one embodiment, the first capsule contains a first color and the second capsule contains a second color (e.g., in order to identify the components of the fill). In one embodiment, the color of the compressed core is one color (e.g., white) and the color of the capsule is non-white (e.g., red, blue, pink, or green). In the embodiment where two liquid filled capsules are on opposite faces of the tablet, the liquid filled capsules are offset from the center. This offset configuration is advantageous in the fact that a multiple capsules can be placed into a tablet, but yet the tablet can retain an overall thickness that is less than the thickness when the two capsules placed on top of one another.

In one embodiment, the surface of the tablet is substantially free of capsules (e.g., the capsules are substantially or completely surrounded by compressed core). The liquid fill capsules may be purchased or made by any method known in the art. Examples of machines for making liquid filled capsules include, but are not limited to, Liqfil Super 40 (Shionogi Qualicaps in Whitsett, N.J.), (Capsugel's (Greenwood, S.C.) Liquid Encapsulation Microspray Sealing (LEMS) production-scale machine. In one embodiment, the liquid fill capsules may be produced using a drop formation process, as shown in U.S. Pat. No. 5,330,835. In another embodiment, the liquid fill capsules may be produced using a fluid injector process as shown in US Patent publication 20050161844 or a process for producing multilayered liquid fill capsules may be used, as shown in U.S. Pat. No. 6,426,089. In another embodiment, the liquid fill capsules may be manufactured wherein the shell is cast into ribbons or sheets as described in U.S. Pat. No. 6,949,256.

### Pharmaceutically Active Agent

The tablet of the present invention includes at least one pharmaceutically active agent. What is meant by a “pharmaceutically active agent” is an agent (e.g., a compound) that is permitted or approved by the U.S. Food and Drug Administration, European Medicines Agency, or any successor entity thereof, for the oral treatment of a condition or disease. Suitable pharmaceutically active agents include, but are not limited to, analgesics, anti-inflammatory agents, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, nicotine, and sedatives.

Examples of suitable gastrointestinal treating agents include, but are not limited to: antacids such as aluminum-containing pharmaceutically active agents (e.g., aluminum carbonate, aluminum hydroxide, dihydroxyaluminum sodium carbonate, and aluminum phosphate), bicarbonate-containing pharmaceutically active agents, bismuth-containing pharmaceutically active agents (e.g., bismuth aluminate, bismuth carbonate, bismuth subcarbonate, bismuth subgallate, and bismuth subnitrate), calcium-containing pharmaceutically active agents (e.g., calcium carbonate), glycine, magnesium-containing pharmaceutically active agents (e.g., magaldrate, magnesium aluminosilicates, magnesium carbonate, magnesium glycinate, magnesium hydroxide, magnesium oxide, and magnesium trisilicate), phosphate-containing pharmaceutically active agents (e.g., aluminum phosphate and calcium phosphate), potassium-containing pharmaceutically active agents (e.g., potassium bicarbonate), sodium-containing pharmaceutically active agents (e.g., sodium bicarbonate), and silicates; laxatives such as stool softeners (e.g., docusate) and stimulant laxatives (e.g., bisacodyl); H2 receptor antagonists, such as famotidine, ranitidine, cimetadine, and nizatidine; proton pump inhibitors such as omeprazole and lansoprazole; gastrointestinal cytoprotectives, such as sucraflate and misoprostol; gastrointestinal prokinetics such as prucalopride; antibiotics for H. pylori, such as clarithromycin, amoxicillin, tetracycline, and metronidazole; antidiarrheals, such as bismuth subsalicylate, kaolin, diphenoxylate, and loperamide; glycopyrrolate; analgesics, such as mesalamine; antiemetics such as ondansetron, cyclizine, diphenyhydroamine, dimenhydrinate, meclizine, promethazine, and hydroxyzine; probiotic bacteria including but not limited to lactobacilli; lactase; racecadotril; and antiflatulents such as polydimethylsiloxanes (e.g., dimethicone and simethicone, including those disclosed in U.S. Pat. Nos. 4,906,478, 5,275,822, and 6,103,260); isomers thereof; and pharmaceutically acceptable salts and prodrugs (e.g., esters) thereof.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof; and pharmaceutically acceptable salts and prodrugs thereof.

Examples of muscle relaxants include, but are not limited to, cyclobenzaprine and chlorzoxazone metaxalone, and orphenadrine, methocarbamol; isomers thereof; and pharmaceutically acceptable salts and prodrugs thereof.

Examples of stimulants include, but are not limited to, caffeine.

Examples of sedatives include, but are not limited to sleep aids such as antihistamines (e.g., diphenhydramine), eszopiclone, and zolpidem, and pharmaceutically acceptable salts and prodrugs thereof.

Examples of appetite suppressants include, but are not limited to, phenylpropanolamine, phentermine, and diethylcathinone, and pharmaceutically acceptable salts and prodrugs thereof

Examples of anesthetics (e.g., for the treatment of sore throat) include, but are not limited to dyclonene, benzocaine, and pectin and pharmaceutically acceptable salts and prodrugs thereof.

Examples of suitable statins include but are not limited to atorvastin, rosuvastatin, fluvastatin, lovastatin, simvustatin, atorvastatin, pravastatin and pharmaceutically acceptable salts and prodrugs thereof.

In one embodiment, the pharmaceutically active agent included within the tablet is selected from phenylephrine, dextromethorphan, pseudoephedrine, acetaminophen, ibuprofen, ketoprofen, loperamide, famotidine, calcium carbonate, simethicone, and menthol, and pharmaceutically acceptable salts and prodrugs thereof.

In one embodiment, the pharmaceutically active agent is selected from phenylephrine, dextromethorphan, pseudoephedrine, chlorpheniramine, methocarbomal, to chlophedianol, ascorbic acid, menthol, pectin, dyclonine, and benzocaine, and pharmaceutically acceptable salts and prodrugs thereof.

As discussed above, the pharmaceutically active agents of the present invention may also be present in the form of pharmaceutically acceptable salts, such as acidic/anionic or basic/cationic salts. Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Pharmaceutically acceptable basic/cationic salts include, and are not limited to aluminum, benzathine, calcium, chloroprocaine, choline, diethanolamine, ethylenediamine, lithium, magnesium, meglumine, potassium, procaine, sodium and zinc.

As discussed above, the pharmaceutically active agents of the present invention may also be present in the form of prodrugs of the pharmaceutically active agents. In general, such prodrugs will be functional derivatives of the pharmaceutically active agent, which are readily convertible in vivo into the required pharmaceutically active agent. Conventional procedures for the selection and preparation of suitable prodrug derivatives are described, for example, in “Design of Prodrugs”, ed. H. Bundgaard, Elsevier, 1985. In addition to salts, the invention provides the esters, amides, and other protected or derivatized forms of the described compounds.

Where the pharmaceutically active agents according to this invention have at least one chiral center, they may accordingly exist as enantiomers. Where the pharmaceutically active agents possess two or more chiral centers, they may additionally exist as diastereomers. It is to be understood that all such isomers and mixtures thereof are encompassed within the scope of the present invention. Furthermore, some of the crystalline forms for the pharmaceutically active agents may exist as polymorphs and as such are intended to be included in the present invention. In addition, some of the pharmaceutically active agents may form solvates with water (e.g., hydrates) or common organic solvents, and such solvates are also intended to be encompassed within the scope of this invention.

In one embodiment, the pharmaceutically active agent or agents are present in the tablet in a therapeutically effective amount, which is an amount that produces the desired therapeutic response upon oral administration and can be readily determined by one skilled in the art. In determining such amounts, the particular pharmaceutically active agent being administered, the bioavailability characteristics of the pharmaceutically active agent, the dose regime, the age and weight of the patient, and other factors must be considered, as known in the art.

The pharmaceutically active agent may be present in various forms. For example, the pharmaceutically active agent may be dispersed at the molecular level, e.g. melted, within the tablet, or may be in the form of particles, which in turn may be coated or uncoated. If the pharmaceutically active agent is in form of particles, the particles (whether coated or uncoated) typically have an average particle size of from about 1 to about 2000 microns. In one embodiment, such particles are crystals having an average particle size of from about 1 to about 300 microns. In another embodiment, the particles are granules or pellets having an average particle size of from about 50 to about 2000 microns, such as from about 50 to about 1000 microns, such as from about 100 to about 800 microns.

In one embodiment, the pharmaceutically active agent with a larger dose is contained in the compressed core while the pharmaceutically active agent with a smaller dose into the liquid filled capsule portion. In one embodiment, an pharmaceutically active agent with at least a 100 mg dose, e.g. at least a 200 mg dose, is placed into the compressed core and an pharmaceutically active agent with a dose of less than 50 mg, e.g., less than 30 mg, is placed into the liquid filled capsule. In one embodiment, a pharmaceutically active agent which is only available as a liquid at room temperature and pressure is present in the liquid filled capsule. In one embodiment, simethicone is present in the liquid filled capsule. In one particular embodiment, the compressed core includes a gastrointestinal active ingredient such as calcium carbonate, aluminum hydroxide, magnesium hydroxide, famotidine, cimetadine, bisacodyl, domperidone, loperamide or loperamide oxide and the liquid filled capsule includes simethicone.

If the pharmaceutically active agent has an objectionable taste, the pharmaceutically active agent may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked pharmaceutically active agents may also be employed. For example, acetaminophen particles, which are encapsulated with ethylcellulose or other polymers by a coaccervation process, may be used in the present invention. Coaccervation-encapsulated acetaminophen may be purchased commercially from Eurand America, Inc. (Vandalia, Ohio) or from Circa Inc. (Dayton, Ohio).

The pharmaceutically active agent may be present in pure crystal form or in a granulated form prior to the addition of the taste masking coating. Granulation techniques may be used to improve the flow characteristics or particle size of the pharmaceutically active agents to make it more suitable for compression or subsequent coating. Suitable binders for making the granulation include but are not limited to starch, polyvinylpyrrolidone, polymethacrylates, hydroxypropylmethylcellulose, and hydroxypropylcellulose. The particles including pharmaceutically active agent(s) may be made by cogranulating the pharmaceutically active agent(s) with suitable substrate particles via any of the granulation methods known in the art. Examples of such granulation method include, but are not limited to, high sheer wet granulation and fluid bed granulation such as rotary fluid bed granulation, the details of which are disclosed in, “The Theory and Practice of Industrial Pharmacy, 3^{rd} edition”, Chapter 11, Lachman, Leon et. al, 1986.

In one embodiment, the core includes gel-coated liquid filled beads, which may contain a flavorant, an pharmaceutically active agent or mixtures thereof. In one embodiment the gel-filled beads are coated with materials that include, but not limited to, hydrocolloids (such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches, and mixtures thereof, and a plasticizer (such as propylene glycol, glycerin or mixtures thereof). Since, in one embodiment, the tablet disclosed herein does not undergo a compression step, the gel-coated liquid filled beads are less likely break.

In one embodiment, the tablet incorporates modified release coated particles (e.g., particles containing at least one pharmaceutically active agent that convey modified release properties of such agent). As used herein, “modified release” shall apply to the altered release or dissolution of the active agent in a dissolution medium, such as gastrointestinal fluids. Types of modified release include, but are not limited to, extended release or delayed release. In general, modified release tablets are formulated to make the active agents(s) available over an extended period of time after ingestion, which thereby allows for a reduction in dosing frequency compared to the dosing of the same active agent(s) in a conventional tablet. Modified release tablets also permit the use of active agent combinations wherein the duration of one pharmaceutically active agent may differ from the duration of another pharmaceutically active agent. In one embodiment the tablet contains one pharmaceutically active agent that is released in an immediate release manner and an additional active agent or a second portion of the same active agent as the first that is modified release.

In one embodiment, at least one pharmaceutically active agent, or a portion of the pharmaceutically active agent in the compressed core, is coated with a modified release coating.

In one embodiment, the compressed core releases a pharmaceutically active agent in a modified release manner and the liquid filled capsule releases a pharmaceutically active agent in an immediate release manner In one embodiment, the compressed core includes a modified release matrix. In one embodiment the compressed core is coated with a modified release coating prior to the addition of the liquid filled capsule. In one embodiment the compressed core is coated with an immediate release coating prior to the addition of the liquid filled capsule. In one embodiment the compressed core including the liquid filled capsule is subsequently coated with an immediate release coating. In one embodiment the compressed core including the liquid filled capsule is subsequently coated with an immediate release coating.

In one embodiment the liquid filled capsule includes a modified release coating. In one embodiment, the modified release coating displays a release rate which is different from the active ingredient in the compressed core.

Examples of swellable, erodible hydrophilic materials for use as a release modifying excipient for use in the compressed core and/or modified release coating include water swellable cellulose derivatives, polyalkylene glycols, thermoplastic polyalkylene oxides, acrylic polymers, hydrocolloids, clays, and gelling starches. Examples of water swellable cellulose derivatives include sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose. Examples of polyalkylene glycols include polyethylene glycol. Examples of suitable thermoplastic polyalkylene oxides include poly (ethylene oxide). Examples of acrylic polymers include potassium methacrylatedivinylbenzene copolymer, polymethylmethacrylate, and high-molecular weight cross-linked acrylic acid homopolymers and copolymers.

Suitable pH-dependent polymers for use as release-modifying excipients for use in the compressed core, in the liquid filled capsule, or in a modified release coating include: enteric cellulose derivatives such as hydroxypropyl methylcellulose phthalate, hydroxypropyl methylcellulose acetate succinate, and cellulose acetate phthalate; natural resins such as shellac and zein; enteric acetate derivatives such as polyvinylacetate phthalate, cellulose acetate phthalate, and acetaldehyde dimethylcellulose acetate; and enteric acrylate derivatives such as for example polymethacrylate-based polymers such as poly(methacrylic acid, methyl methacrylate) 1:2 (available from Rohm Pharma GmbH under the tradename EUDRAGIT S) and poly(methacrylic acid, methyl methacrylate) 1:1 (available from Rohm Pharma GmbH under the tradename EUDRAGIT L).

In one embodiment the pharmaceutically active agent is coated with a combination of a water insoluble film forming polymer (such as but not limited to cellulose acetate or ethylcellulose) and a water soluble polymer (such as but not limited to povidone, polymethacrylic co-polymers such as those sold under the tradename Eudragit E-100 from Rohm America, and hydroxypropylcellulose). In this embodiment, the ratio of water insoluble film forming polymer to water soluble polymer is from about 50 to about 95 percent of water insoluble polymer and from about 5 to about 50 percent of water soluble polymer, and the weight percent of the coating by weight of the coated taste-masked particle is from about 5 percent to about 40 percent.

In one embodiment one or more pharmaceutically active agents or a portion of the pharmaceutically active agent may be bound to an ion exchange resin for the purposes of taste-masking the pharmaceutically active agent or delivering the active in a modified release manner.

In one embodiment, the pharmaceutically active agent is capable of dissolution upon contact with a fluid such as water, stomach acid, intestinal fluid or the like. In one embodiment, the dissolution characteristics of the pharmaceutically active agent within the tablet meets USP specifications for immediate release tablets including the pharmaceutically active agent. For example, for acetaminophen tablets, USP 24 specifies that in pH 5.8 phosphate buffer, using USP apparatus 2 (paddles) at 50 rpm, at least 80% of the acetaminophen contained in the tablet is released there from within 30 minutes after dosing, and for ibuprofen tablets, USP 24 specifies that in pH 7.2 phosphate buffer, using USP apparatus 2 (paddles) at 50 rpm, at least 80% of the ibuprofen contained in the tablet is released there from within 60 minutes after dosing. See USP 24, 2000 Version, 19-20 and 856 (1999). In another embodiment, the dissolution characteristics of the pharmaceutically active agent are modified: e.g. controlled, sustained, extended, retarded, prolonged, delayed and the like.

### Tablets Coatings

In one embodiment, the tablet includes an additional outer coating (e.g., a translucent coating such as a clear coating). Suitable materials for translucent coatings include, but are not limited to, hypromellose, hydroxypropylcellulose, starch, polyvinyl alcohol, polyethylene glycol, polyvinylalcohol and polyethylene glycol mixtures and copolymers, and mixtures thereof.

In one embodiment the tablet is dipped coated in gelatin or a starch based coating prior to the addition of the liquid filled capsule. In one embodiment the tablet is dip coated in a gelatin or starch based coating after the addition of the liquid filled capsule.

### Use of Tablet

The tablets may be used as swallowable, chewable, or orally disintegrating tablets to administer the pharmaceutically active agent. In case of chewable or orally disintegrating tablets, the capsule can be adapted to be chewable, swallowable, or orally dissolvable.

In one embodiment, the present invention features a method of treating an ailment, the method including orally administering the above-described tablet wherein the tablet includes an amount of the pharmaceutically active agent effective to treat the ailment. Examples of such ailments include, but are not limited to, pain (such as headaches, migraines, sore throat, cramps, back aches and muscle aches), fever, inflammation, upper respiratory disorders (such as cough and congestion), infections (such as bacterial and viral infections), depression, diabetes, obesity, cardiovascular disorders (such as high cholesterol, triglycerides, and blood pressure), gastrointestinal disorders (such as nausea, diarrhea, irritable bowel syndrome and gas), sleep disorders, osteoporosis, and nicotine dependence.

In one embodiment, the method is for the treatment of an upper respiratory disorder, wherein the pharmaceutically active agent is selected from the group of phenylephrine, cetirizine, loratadine, fexofenadine, diphenhydramine, dextromethorphan, chlorpheniramine, chlophedianol, and pseudoephedrine.

In this embodiment, the “unit dose” is typically accompanied by dosing directions, which instruct the patient to take an amount of the pharmaceutically active agent that may be a multiple of the unit dose depending on, e.g., the age or weight of the patient. Typically the unit dose volume will contain an amount of pharmaceutically active agent that is therapeutically effective for the smallest patient. For example, suitable unit dose volumes may include one tablet).

In one embodiment, the tablet is used as a treatment for allergic conditions associated with decongestion, wherein the liquid filled capsule includes a non-sedating antihistamine and the compressed portion includes a decongestant. In one version of this embodiment, the non-sedating antihistamine is cetirizine and the decongestant is phenylephrine or pseudoephedrine. In one version of this embodiment, the cetirizine is delivered as a 12 to 24 hour duration immediate release dose and the decongestant is delivered in an extended or sustained release manner for 12 to 24 hours.

In one embodiment, the liquid filled capsule contains a unique identifier or identifying property to prevent counterfeiting of the tablet. In this embodiment, the identifier may be a color, printing, or a microrelief either on the surface or as part of the liquid filled capsule. Other identifiers in the liquid filled capsule surface may include a suspended flake, particle, UV light pigment or other effect pigment. In certain embodiments wherein the surface of a single liquid filled capsule is visible on 2 faces of the tablet, the tablet may be held to a light such that as light passes through the tablet, the identifying property is visible. In another embodiment, the liquid filled capsule includes particles or sparkled flakes that display separate effects or diffract or reflect light at different angles or under light. In embodiments where particle or flakes are added to the shell, the particles or flakes are made of materials such as but are not limited to titanium dioxide, aluminum lakes, magnesium lakes, calcium lakes, mica, pearlescent colors, fluorescent materials, and flavorants.

### Examples

Specific embodiments of the present invention are illustrated by way of the following examples. This invention is not confined to the specific limitations set forth in these examples.

### Example 1

### Preparation of Compressed Caplet Core Granulation

4.0 kg of the materials in Table 1 are blended in a Glatt GCPG 5/9 top spray fluid bed coating unit (Glatt, Ramsey, N.J.). A solution of 27.3% weight by weight of phenylephrine hydrochloride in purified water USP is sprayed onto the granulation materials in the Glatt 5/9 at approximately 10 g/minute and a product temperature of 28-32° C. and an atomization air pressure of 2 bars. A granulating solution of 7.0 percent weight by weight of cornstarch NF in purified water is sprayed onto the blend inside of the fluid bed granulator at a product temperature of 25-30° C. at approximately 20 g/minute and dried to a temperature of 35° C.

TABLE 1
Granulation Materials

| | Weight |
|---|---|
| Granulation Material | Percent |
| Acetaminophen | 86.4 |
| USP | |
| Powdered Cellulose | 5.6 |
| NF | |
| Microcrystalline | 5.3 |
| Cellulose | |
| Pregelatinized | 1.9 |
| Starch NF | |
| Sodium Starch | 0.8 |
| Glycolate NF | |

### Example 2

### Preparation of Blend for Compression

2475.5 g of the granulation prepared in Example 1 are placed into a twin-shell blender. 16.1 g of colloidal silicon dioxide NF, 54.3 g of stearic acid NF, 889.0 g of microcrystalline cellulose NF, and 65.1 g of sodium starch glycolate NF are added to the blend and blended end-over end for 10 minutes, and discharged into a plastic bag.

### Example 3

### Preparation of Compressed Core

The blend from Example 2 is compressed on a Manesty rotary lab tablet press (Manesty, Knowsley, Merseyside, UK) using caplet tooling of 0.750 inches×0.25 inches×0.075 inches at a hardness of 11.1 to 15.6 kiloponds, a weight of 575 to 609 mg, and a thickness of 6.01 mm to 6.21 mm. The caplet tooling has a cavity that extends through the short axis of the caplet.

### Example 4

### Preparation of Gray Film Coating Solution

340 g of sterile water for irrigation are added to a 2-liter stainless steel vessel. A Lightning laboratory mixer is set to 50 RPM and 85.0 grams of hypromellose based film coating polymer containing gray colorant (commercially available from the Colorcon Corporation, Exton, Pa. as Opadry® Gray) are added and mixed for 45 minutes.

### Example 5

### Gray Film Coating of Compressed Cores

3.0 kg of caplets from each of Example 3 are added to a 24-inch vented Accla Cota coating pan (Thomas Engineering Inc, Hoffman Estates, Ill.). The batch is spray coated with a spray rate of approximately 12 grams per minute, about 14 RPM, an inlet air temperature of about 85° C., and an atomization air pressure of about 55 psi. 405 grams of the coating solution are sprayed, which are equivalent to 81 g of dried coating or about a 2.7% weight gain.

### Example 6

### Preparation of Liquid Filled Capsules

### Example 6A

### Preparation of Liquid Fill Solution

5 g of citric acid and 1 gram of peppermint flavor is dissolved into 20 g of purified water. While stirring at 50 RPM using a lab based propeller-type mixer, the citric acid and flavor mixture is stirred into 974 grams of olive oil. This solution is held at approximately 25° C.

### Example 6B

### Preparation of Gelatin Solution for Capsule Shell

1440 g of sterile water for irrigation are added to a 3-liter stainless steel vessel. Utilizing a laboratory mixer set at 80 RPM while stirring, 550 g of Gelatin 240 Bloom (Commercially available from Gelita AG, in Eberbach, Germany) is added to the water. Then, the mixture is heated to 65° C., and 28 grams of Opatint Red DD15130 (Commercially available from the Colorcon Corporation) is added while mixing. Then the solution is mixed at low speed for 4 hours (at ambient pressure) to deaerate while the tank is maintained at a solution temperature of about 60° C.

### Example 6C

### Preparation of Liquid Filled Capsules

The liquid fill solution from Example 6A is pumped through a gear type pump at approx 10 grams per minute while the gelatin solution from Example 6B is pumped through a separate gear pump. The two nozzles are placed on top of one another with sizes of 0.7 mm and 1.2 mm for the liquid fill solution and the gelatin solution, respectively. The two solutions are simultaneously pumped through the two nozzles into a reciculating bath containing approximately 400 g of neobee oil that is being recirculated into a 5 Liter batch of neobee oil. The pumped solutions are delivered into a pulsating stream, which is pulsed using a vibratory mechanism, and which is then directed into the recirculating neobee oil, upon which the gelatin solution solidifies around the liquid fill solution. The size of the capsule is regulated by an external rotating diaphragm located above the nozzle, which actuates the stream of the two solutions, as well as the size of the nozzle. The size is then controlled by speed of the diaphragm, which is adjusted using an external controller. The capsules are circulated through the oil bath into straining filter of approximately 20 US mesh. They are then allowed to dry for 48 hours at 25° C.

### Example 7

### Preparation of Compressed Cores Containing Liquid Filled Capsule

The coated compressed cores from Example 5 are combined with the liquid filled capsules from Example 6 by manually inserting the capsule into the cavity in the coated compressed core as follows. First, 340 g of sterile water for irrigation are added to a 2-liter stainless steel vessel. A laboratory mixer is then set to 50 RPM, and 85 grams of hypromellose film polymer (commercially available from the Dow Corporation, Midland, Mich. as Methocel E5) are added and mixed for 45 minutes. Approximately 0.25 mL of solution is added manually to the cavity portion of each coated tablet from Example 5. The liquid filled capsule from Example 6C is then added to the cavity and placed into an oven at 60° C. for approximately 24 hours to dry.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A tablet comprising a compressed core and a liquid filled capsule, wherein said compressed core comprises a first pharmaceutically active agent, the compressed core comprises a cavity exposed on the surface of said core, and said capsule is contained within said cavity such that a portion of said capsule is visible on said surface of said tablet, wherein said capsule has a diameter of at least 500 microns.

2. The tablet of claim 1, wherein said tablet comprises a plurality of liquid filled capsules wherein the surface of said core comprises one or more cavities and said capsules are contained within said one or more cavities such that a portion of each of said capsules are visible on said surface of said tablet.

3. The tablet of claim 2, wherein said tablet has a first surface and a second surface, wherein said second surface is on the opposite side of said tablet from said first surface, wherein said first surface comprises at least one cavity containing one of said capsules, and wherein said second surface comprises at least one cavity containing one of said capsules.

4. The tablet of claim 1, wherein the compressed core has a density of at least 0.9 g/cc.

5. The tablet of claim 1, wherein said capsule has a diameter of from about 500 to about 10,000 microns.

6. The tablet of claim 1, wherein said capsule comprises ingredients selected from the group consisting of a second pharmaceutically active agent, a lubricant, a salivation-inducing agent, a warming sensate, a cooling sensate, and flavors.

7. The tablet of claim 6, wherein said capsule comprises a lubricant selected from the group consisting of fatty acids, olive oil, mineral oil, and cocoa butter.

8. The tablet of claim 6, wherein said capsule comprises a salivation-inducing agent.

9. The tablet of claim 6, wherein said capsule comprises a second pharmaceutically active agent.

10. The tablet of claim 9, wherein said first pharmaceutically active agent is selected from antihistamines, decongestants, and expectorants and said second pharmaceutically active agent is an anesthetic.

11. The tablet of claim 1, wherein the tablet comprises an additional translucent coating.

12. A method of administering a pharmaceutically active agent to a subject, said method comprising orally administering the tablet of claim 1.

13. The method of claim 12, wherein said tablet comprises a plurality of liquid filled capsules wherein the surface of said core comprises one or more cavities and said capsules are contained within said one or more cavities such that a portion of each of said capsules are visible on said surface of said tablet.

14. The tablet of claim 13, wherein said tablet has a first surface and a second surface, wherein said second surface is on the opposite side of said tablet from said first surface, wherein said first surface comprises at least one cavity containing one of said capsules, and wherein said second surface comprises at least one cavity containing one of said capsules.

15. The tablet of claim 12, wherein said capsule comprises ingredients selected from the group consisting of a second pharmaceutically active agent, a lubricant, a salivation-inducing agent, a warming sensate, a cooling sensate, and flavors.

16. The method of claim 15, wherein said capsule comprises a second pharmaceutically active agent.

17. The method of claim 16, wherein said first pharmaceutically active agent is selected from antihistamines, decongestants, and expectorants and said second pharmaceutically active agent is an anesthetic.

18. A method of manufacturing a tablet of claim 1, said method comprising the steps of:
(a) adding a powder comprising a pharmaceutically-acceptable carrier and said first pharmaceutically active agent to a tablet die;
(b) compressing said powder within said tablet die to form said compressed core; and
(c) inserting said capsule into said cavity in said compressed core to form said tablet.

19. A method of claim 18, wherein said cavity is formed by said tablet die during the compression of step (b).

20. The method of claim 18, wherein said method further comprises the step of applying an adhesive to said cavity prior to step (c).
